(19) United States
(12) Patent Application Publication
(10) Pub. No.: US 2014/0296710 A1
(43) Pub. Date: Oct. 2, 2014

(54) ULTRASONIC DIAGNOSTIC APPARATUS

(71) Applicant: Konica Minolta, Inc., Tokyo (JP)

(72) Inventors: Masashi KUNITA, Kanagawa (JP); Takashi SAKAI, Kanagawa (JP)

(73) Assignee: Konica Minolta, Inc., Tokyo (JP)

(21) Appl. No.: 14/243,039

(22) Filed: Apr. 2, 2014

(30) Foreign Application Priority Data
Apr. 2, 2013 (JP) 2013-076559

(51) Int. Cl. A61B 8/00

(52) U.S. Cl. CPC A61B 8/463
USPC 600/440

(57) ABSTRACT

An ultrasonic diagnostic apparatus is provided that improves ultrasonogram visibility without requiring an operator's effort in simultaneous display of multiple ultrasonograms. Ultrasonic diagnostic apparatus (100) includes: display range selection section (62) that sets a first display range on a first ultrasonogram and sets a second display range on a second ultrasonogram on the basis of the first display range set on the first ultrasonogram; and screen layout setting section (63) that sets a screen layout such that an ultrasonogram display area on a display screen is vertically or horizontally divided into a first display area and a second display area. Screen layout setting section (63) sets a screen layout such that the ultrasonogram display area is divided in a direction that allows the first ultrasonogram in the first display range to be displayed larger in the first display area.

### CROSS REFERENCE TO RELATED APPLICATIONS

This application is entitled and claims the benefit of Japanese Patent Application No. 2013-076559, filed on Apr. 2, 2013, the disclosure of which including the specification, drawings and abstract is incorporated herein by reference in its entirety.

### TECHNICAL FIELD

The present invention relates to an ultrasonic diagnostic apparatus.

### BACKGROUND ART

An ultrasonic diagnostic apparatus is connected to a probe and a display to transmit and receive ultrasound to and from a subject via the probe, thereby imaging the inside of the subject as an ultrasonogram.

For example, PTL 1 proposes an ultrasonic diagnostic apparatus that simultaneously displays an ultrasonogram associated with a past diagnosis (hereinafter referred to as a “recorded image”) and an ultrasonogram associated with a current diagnosis (hereinafter referred to as a “real-time image”) on a display.

### CITATION LIST

### Patent Literature

PTL 1

Japanese Patent Application Laid-Open No. 2000-175912

### SUMMARY OF INVENTION

### Technical Problem

The aspect ratio of an ultrasonogram varies with factors such as the depth of a site to be diagnosed from the body surface. However, the ultrasonic diagnostic apparatus disclosed in PTL 1 has a fixed screen layout. This poses the problem of low visibility of ultrasonograms displayed on the display, depending on their aspect ratio.

The present invention solves the foregoing problem pertinent in the art, and an object thereof is to provide an ultrasonic diagnostic apparatus that enables setting an appropriate screen layout with uncompromised ultrasonogram visibility while requiring no effort of an operator.

### Solution to Problem

An ultrasonic diagnostic apparatus of the present invention that is connectable to a display and allows a first ultrasonogram and a second ultrasonogram to be simultaneously displayed on the display includes: a display range selection section that sets a first display range on the first ultrasonogram and sets a second display range on the second ultrasonogram on the basis of the first display range set on the first ultrasonogram; a screen layout setting section that sets a screen layout such that an ultrasonogram display area on a display screen is vertically or horizontally divided into a first display area and a second display area; and an image combining and outputting section that generates a display screen on which the first ultrasonogram in the first display range is assigned to the first display area and the second ultrasonogram in the second display range is assigned to the second display area, and that outputs the generated display screen to the display. The screen layout setting section sets a screen layout such that the ultrasonogram display area is divided in a direction that allows the first ultrasonogram in the first display range to be displayed larger in the first display area.

### Advantageous Effects of Invention

The ultrasonic diagnostic apparatus of the present invention enables setting an appropriate screen layout with uncompromised ultrasonogram visibility while requiring no effort of an operator.

### BRIEF DESCRIPTION OF DRAWINGS

FIG. 1 is a schematic functional block diagram of an ultrasonic diagnostic apparatus according to Embodiment 1 of the present invention;

FIG. 2 is a functional block diagram of a display processing section of the ultrasonic diagnostic apparatus according to Embodiment 1 of the present invention;

FIG. 3 is an operational flow diagram illustrating operations of the ultrasonic diagnostic apparatus according to Embodiment 1 of the present invention;

FIGS. 4A to 4F illustrate a first example of screen layout setting of the ultrasonic diagnostic apparatus according to Embodiment 1 of the present invention;

FIGS. 5A and 5B illustrate a variation of the first example of screen layout setting of the ultrasonic diagnostic apparatus according to Embodiment 1 of the present invention;

FIGS. 6A to 6C illustrate a second example of screen layout setting of the ultrasonic diagnostic apparatus according to Embodiment 1 of the present invention; and

FIGS. 7A to 7C illustrate a third example of screen layout setting of the ultrasonic diagnostic apparatus according to Embodiment 1 of the present invention.

### DESCRIPTION OF EMBODIMENTS

An embodiment of the present invention will be described in detail below with reference to the accompanying drawings.

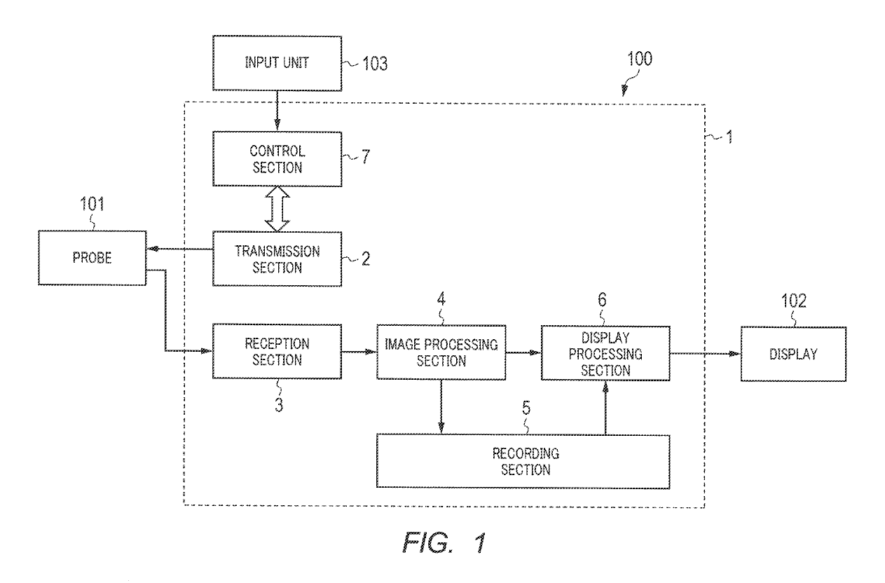

As illustrated in FIG. 1, ultrasonic diagnostic apparatus 100 is electrically connectable with probe 101, display 102, and input unit 103. Ultrasonic diagnostic apparatus 100 includes controller 1, which includes transmission section 2, reception section 3, image processing section 4, recording section 5, display processing section 6, and control section 7.

### Probe 101

Probe 101 has lineally arranged transducers, for example. Probe 101 converts pulsed or continuous-wave electric signals (hereinafter referred to as “transmitted electric signals”) supplied from transmission section 2 into pulsed or continuous-wave ultrasonic waves. While being in contact with the skin surface of a subject, probe 101 transmits, to the inside of the subject, an ultrasonic beam comprised of the ultrasonic waves emitted from the transducers.

Probe 101 then receives reflected ultrasonic waves from the inside of the subject. Probe 101 converts the reflected ultrasonic waves into respective electric signals (hereinafter referred to as “received electric signals”) with the transducers and supplies the received electric signals to reception section 3.

### Display 102

Display 102 is a so-called monitor, which displays thereon output from display processing section 6 as a display screen.

### Input Unit 103

Input unit 103 is used by an operator to make various settings on ultrasonic diagnostic apparatus 100, including registering patient information, selecting a diagnostic mode (such as B-mode or color doppler mode), setting the display depth, setting a region of interest (hereinafter referred to as an “ROI”), and setting a body mark. Input unit 103 can also be used to set a simultaneous display mode of multiple ultrasonograms on display 102, to set recording of ultrasonograms and associated diagnostic information in recording section 5, and to select an ultrasonogram recorded in recording section 5.

### Transmission Section 2

Transmission section 2 connects with probe 101 and performs a transmission process. The transmission process includes generating transmission control signals for controlling the transmission of an ultrasonic beam from probe 101, and supplying, to probe 101, pulsed or continuous-wave transmitted electric signals generated on the basis of the transmission control signals.

### Reception Section 3

Reception section 3 performs a reception process, which includes amplifying received electric signals from probe 101 and subjecting the amplified signals to A/D conversion to thereby generate received signals. Reception section 3 then outputs the received signals to image processing section 4. The transmission process in transmission section 2 and the reception process in reception section 3 are continuously repeated to form frames, each consisting of multiple received signals. A frame herein refers to a set of received signals required for forming an ultrasonogram, signals processed for forming an ultrasonogram on the basis of the set of received signals, or an ultrasonogram formed on the basis of the set of received signals.

### Image Processing Section 4

Image processing section 4 has the same configuration as in a general ultrasonic diagnostic apparatus and is therefore not illustrated in detail in FIG. 1. Image processing section 4 includes components such as various filters, a wave detector, a logarithmic amplifier, a scan converter, and other signal or image processors. Image processing section 4 converts received signals in a frame into respective luminance signals corresponding to the intensities of the received signals and converts the luminance signals into coordinates in an orthogonal coordinate system, thereby generating an ultrasonogram. Image processing section 4 outputs the generated ultrasonogram to recording section 5 and display processing section 6.

### Recording Section 5

Recording section 5 records, according to settings made through input unit 103, an ultrasonogram obtained in a diagnosis. Also according to settings made through input unit 103, recording section 5 outputs a recorded ultrasonogram and associated diagnostic information to display processing section 6.

### Display Processing Section 6

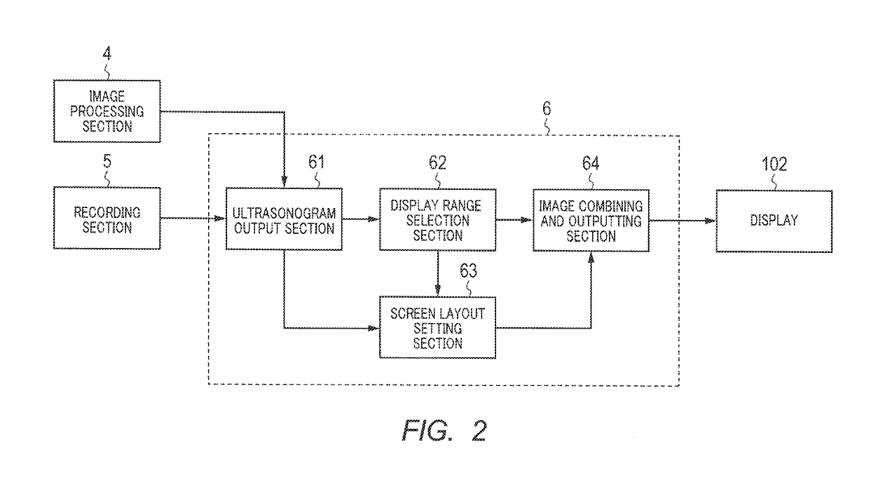

As illustrated in FIG. 2, display processing section 6 includes ultrasonogram output section 61, display range selection section 62, screen layout setting section 63, and image combining and outputting section 64.

### Ultrasonogram Output Section 61

When the simultaneous display mode of multiple ultrasonograms on display 102 is not set by the operator through input unit 103, ultrasonogram output section 61 outputs a real-time image from image processing section 4 to screen layout setting section 63.

When the simultaneous display mode of multiple ultrasonograms on display 102 is set by the operator through input unit 103, ultrasonogram output section 61 outputs, to display range selection section 62, a real-time image and a recorded image in recording section 5, selected by the operator through input unit 103.

### Display Range Selection Section 62

On the basis of a display range on a real-time image set by the operator through input unit 103 (hereinafter referred to as a “first display range”), display range selection section 62 sets a display range on a recorded image (hereinafter referred to as a “second display range”). Specifically, display range selection section 62 sets the second display range representing the same image region as the first display range set on the real-time image: the second display range is located on the recorded image at a position corresponding to the position of the first display range set on the real-time image in the longitudinal direction and the lateral direction orthogonal to the longitudinal direction. The first display range set on the real-time image may cover the entire real-time image or only a desired part of the real-time image designated by the operator.

### Screen Layout Setting Section 63

When the simultaneous display mode of multiple ultrasonograms on display 102 is not set by the operator through input unit 103, screen layout setting section 63 sets a screen layout such that a real-time image is displayed in an ultrasonogram display area on a display screen displayed on display 102.

When the simultaneous display mode of multiple ultrasonograms on display 102 is set by the operator through input unit 103, screen layout setting section 63 sets a screen layout such that the ultrasonogram display area is vertically or horizontally divided into equal areas depending on the number of ultrasonograms to be simultaneously displayed.

This will be described in detail for an exemplary case of simultaneously displaying two ultrasonograms, i.e., a real-time image and a recorded image. It is assumed that the ultrasonogram display area is vertically or horizontally halved, and that the ultrasonogram in the first display range is enlarged to the maximum within one partial area (hereinafter referred to as a “first display area,” whereas the other partial area is referred to as a “second display area”) of the ultrasonogram display area. Screen layout setting section 63 selects a dividing direction that allows the first display range to be displayed larger, and sets a screen layout such that the ultrasonogram display area is divided in the selected dividing direction.

### Image Combining and outputting Section 64

Image combining and outputting section 64 combines ultrasonograms with the ultrasonogram display area according to a screen layout set by screen layout setting section 63, and outputs it to display 102.

Specifically, when the simultaneous display mode of multiple ultrasonograms on display 102 is not set by the operator through input unit 103, image combining and outputting section 64 combines a real-time image with the ultrasonogram display area according to the set screen layout to form a display screen, and displays the display screen on display 102. When the simultaneous display mode of multiple ultrasonograms on display 102 is set by the operator through input unit 103, image combining and outputting section 64 assigns ultrasonograms to their respective partial ultrasonogram display areas to form a display screen, and outputs the display screen to display 102. In the latter case, it is preferable from the viewpoint of ultrasonogram visibility that each ultrasonogram is enlarged to the maximum size within the relevant area and displayed.

### Control Section 7

Control section 7 controls each functional block in controller 1 on the basis of settings made through input unit 103.

### Operations

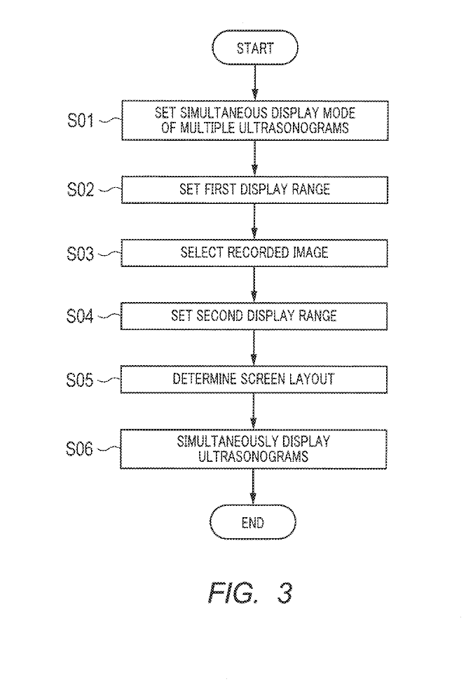

Operations of ultrasonic diagnostic apparatus 100 configured as above, together with the operator's operations, will be described with reference to an operational flow diagram in FIG. 3. The operations of ultrasonic diagnostic apparatus 100 will be described here for the case of simultaneously displaying two images, i.e., a real-time image and a recorded image, on display 102. The operation to obtain the real-time image is the same as in a general ultrasonic diagnostic apparatus and therefore will not be described. It is assumed that the recorded image has been recorded in a past diagnosis. That is, the following describes operations in which data of the two ultrasonograms, i.e., the real-time image in a current diagnosis and the recorded image, is input to display processing section 6 and displayed on display 102.

### Step 1 (S01)

In step 1 (S01), the operator operates input unit 103 to set the simultaneous display mode of two ultrasonograms, i.e., a real-time image and a recorded image.

### Step 2 (S02)

In step 2 (S02), the operator refers to a real-time image displayed on display 102 to set the first display range on the real-time image. The first display range set on the real-time image may cover the entire real-time image or only a desired part of the real-time image designated by the operator.

### Step 3 (S03)

In step 3 (S03), the operator operates input unit 103 to select a desired recorded image in recording section 5. In response to this operation, the selected recorded image is output to display range selection section 62 via ultrasonogram output section 61.

### Step 4 (S04)

In step 4 (S04), display range selection section 62 sets the second display range on the recorded image on the basis of the first display range set on the real-time image. The first display range and the second display range represent the same image region. Display range selection section 62 sets the second display range on the recorded image at a position corresponding to the first display range set on the real-time image in the longitudinal direction and the lateral direction orthogonal to the longitudinal direction.

### Step 5 (S05)

In step 5 (S05), screen layout setting section 63 sets a screen layout such that the ultrasonogram display area is vertically or horizontally divided into equal areas depending on the number of ultrasonograms to be simultaneously displayed.

This will be described in detail for an exemplary case of simultaneously displaying two ultrasonograms, i.e., a real-time image and a recorded image. It is assumed that the ultrasonogram display area is vertically or horizontally halved, and that the ultrasonogram in the first display range is enlarged to the maximum within the first display area of the ultrasonogram display area. Screen layout setting section 63 selects a dividing direction that allows the first display range to be displayed larger, and sets a screen layout such that the ultrasonogram display area is divided in the selected dividing direction.

### Step 6 (S06)

In step 6 (S06), image combining and outputting section 64 assigns the ultrasonograms to their respective partial ultrasonogram display areas according to the screen layout set by screen layout setting section 63 to form a display screen, and outputs the display screen to display 102. If the ultrasonograms of the real-time image and the recorded image to be displayed are not of the same size, image combining and outputting section 64 enlarges the recorded image to match the size of the real-time image.

Thus, the exemplary operations of the ultrasonic diagnostic apparatus of the present invention have been described. Now, specific examples of steps 4 (S04) to 6 (S06) will be described.

### Specific Examples

### First Example

A first example will be described for the case in which the first display range covers the entire real-time image, and the real-time image and the recorded image are ultrasonograms obtained under the same conditions.

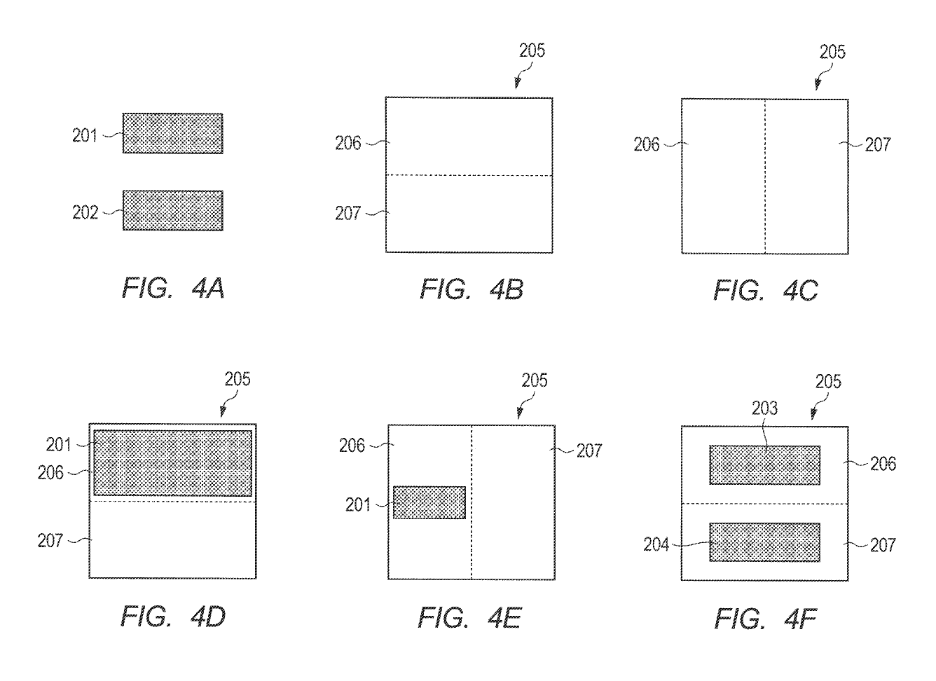

FIG. 4A illustrates real-time image 201 and recorded image 202 to be simultaneously displayed on display 102. As can be seen from FIG. 4A, real-time image 201 is a landscape-oriented ultrasonogram. Recorded image 202, obtained under the same conditions as the real-time image, is also a landscape-oriented ultrasonogram representing the same image region as the real-time image. Such a landscape-oriented image is obtained when, for example, the operator sets a relatively small display depth through input unit 103.

In the first example, first display range 203 covers entire real-time image 201. Since recorded image 202 represents the same image region as real-time image 201, display range selection section 62 also sets second display range 204 to cover entire recorded image 202.

It is then assumed that ultrasonogram display area 205 is vertically halved (i.e., into the upper and lower areas) as illustrated in FIG. 4B or horizontally halved (i.e., into the right and left areas) as illustrated in FIG. 4C to provide first display area 206 and second display area 207. As illustrated in FIGS. 4D and 4E, screen layout setting section 63 enlarges the real-time image of first display range 203 to the maximum within first display area 206.

Screen layout setting section 63 compares the dimension of the enlarged real-time image of first display range 203 in the vertically halved screen (FIG. 4D) with that in the horizontally halved screen (FIG. 4E), and selects the dividing direction providing the larger real-time image. In the example of FIGS. 4A to 4F, as can be seen from FIGS. 4D and 4E, the enlarged real-time image of first display range 203 in FIG. 4D is larger. Screen layout setting section 63 therefore sets a screen layout such that ultrasonogram display area 205 is vertically halved, as illustrated in FIG. 4F.

Image combining and outputting section 64 forms a display screen on which the ultrasonograms of first and second display ranges 203 and 204 are assigned to first and second display areas 206 and 207, respectively. Image combining and outputting section 64 displays the formed display screen on display 102.

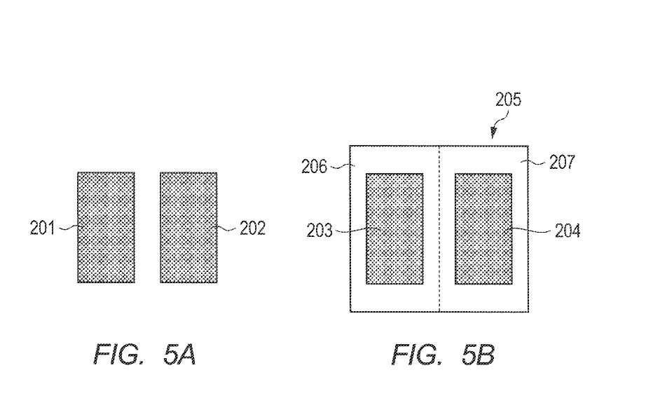

In the case of portrait-oriented ultrasonograms as illustrated in FIG. 5A for the same ultrasonogram display area as ultrasonogram display area 205 in FIG. 4B, screen layout setting section 63 sets the screen layout in the same manner as above. That is, screen layout setting section 63 enlarges the real-time image of first display range 203 to the maximum within first display area 206, and selects the dividing direction providing the larger real-time image of first display range 203.

In the example of FIGS. 5A and 5B, as illustrated in FIG. 5B, the enlarged real-time image of first display range 203 is larger in the horizontally halved screen.

Screen layout setting section 63 therefore sets the horizontally halved screen layout. Such a portrait-oriented image is obtained when, for example, the operator sets a relatively great display depth through input unit 103.

### Second Example

A second example will be described for the case in which the ultrasonogram display area is the same as ultrasonogram display area 205 illustrated in the first example, the first display range covers the entire real-time image, and the real-time image is a landscape-oriented ultrasonogram while the recorded image is a portrait-oriented ultrasonogram.

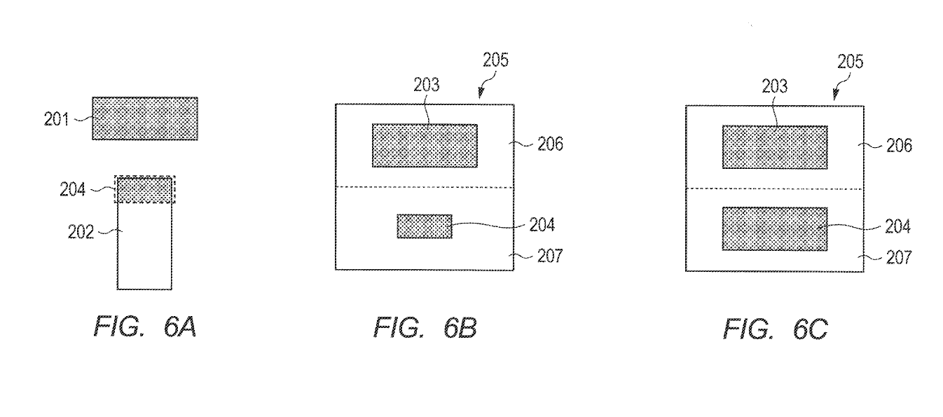

FIGS. 6A illustrates real-time image 201 and recorded image 202 to be simultaneously displayed on display 102. Since recorded image 202 is a portrait-oriented ultrasonogram, second display range 204 on recorded image 202 corresponding to first display range 203 is an area surrounded by a dashed line in FIG. 6A.

As in the first example, it is assumed that ultrasonogram display area 205 is horizontally or vertically halved, and that the real-time image of first display range 203 is enlarged to the maximum within first display area 206. Screen layout setting section 63 selects a dividing direction providing the larger ultrasonogram of first display range 203 and sets the screen layout accordingly.

That is, it is assumed that ultrasonogram display area 205 is vertically halved as illustrated in FIG. 4B or horizontally halved as illustrated in FIG. 4C to provide first display area 206 and second display area 207, and that the real-time image of first display range 203 is enlarged to the maximum within first display area 206. Screen layout setting section 63 selects a dividing direction providing the larger ultrasonogram of first display range 203.

In the example of FIGS. 6A to 6C where real-time image 201 is a landscape-oriented ultrasonogram, screen layout setting section 63 sets the vertically halved screen layout for ultrasonogram display area 205, as illustrated in FIG. 4D.

As illustrated in FIG. 6B, image combining and outputting section 64 assigns first and second display ranges 203 and 204 to first and second display areas 206 and 207, respectively. Since the ultrasonograms of first and second display ranges 203 and 204 are of different sizes, image combining and outputting section 64 adjusts the size of the recorded image of second display range 204 to match the size of the ultrasonogram of first display range 203, as illustrated in FIG. 6C. After this process, image combining and outputting section 64 displays the resulting display screen on display 102.

### Third Example

A third example will be described for the case in which the ultrasonogram display area is the same as ultrasonogram display area 205 illustrated in the first example, and the first display range is an ROI that is set on part of the real-time image.

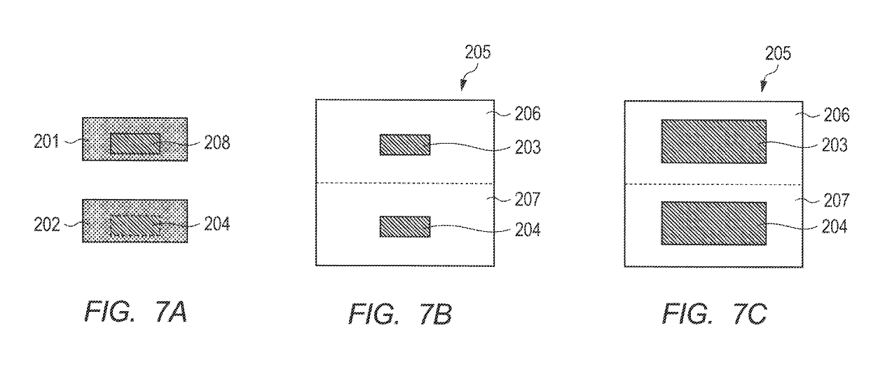

FIG. 7A illustrates real-time image 201 and recorded image 202 to be simultaneously displayed on display 102. ROI 208, which is set on real-time image 201 illustrated in FIG. 7A, is first display range 203. The area surrounded by a dashed line in recorded image 202 in FIG. 7A, corresponding to first display range 203 on real-time image 201, is second display range 204 in recorded image 202.

As in the first example, it is assumed that ultrasonogram display area 205 is horizontally or vertically halved, and that the ultrasonogram of first display range 203 is enlarged to the maximum within first display area 206. Screen layout setting section 63 selects a dividing direction providing the larger ultrasonogram of first display range 203 and sets the screen layout accordingly.

That is, it is assumed that ultrasonogram display area 205 is vertically halved as illustrated in FIG. 4B or horizontally halved as illustrated in FIG. 4C to provide first display area 206 and second display area 207, and that the real-time image of first display range 203 is enlarged to the maximum within first display area 206. Screen layout setting section 63 selects a dividing direction providing the larger real-time image of first display range 203. In the example of FIGS. 7A to 7C where real-time image 201 is a landscape-oriented ultrasonogram, screen layout setting section 63 sets the vertically halved screen layout for ultrasonogram display area 205, as illustrated in FIG. 4D.

As illustrated in FIG. 7B, image combining and outputting section 64 assigns first and second display ranges 203 and 204 to first and second display areas 206 and 207, respectively. Since first and second display ranges 203 and 204 are both partial ultrasonograms of real-time image 201 and recorded image 202, respectively, image combining and outputting section 64 enlarges the ultrasonograms of first and second display ranges 203 and 204 to match their sizes, as illustrated in FIG. 7C. After this process, image combining and outputting section 64 displays the resulting display screen on display 102.

In the above description, the first display range is the ROI set on part of the ultrasonogram. In certain diagnoses, it may be desired to view the region in the set ROI as well as regions around the ROI. In such a case, a region including both the set ROI and a predetermined range around the ROI may be set as the first display range.

### CONCLUSION

The ultrasonic diagnostic apparatus of the present invention thus enables setting an appropriate screen layout on the basis of a first display range set on a first ultrasonogram. Therefore, an appropriate screen layout with uncompromised ultrasonogram visibility can be set while requiring no effort of an operator.

Although Embodiment 1 has been described for the screen layouts such that two ultrasonograms are simultaneously displayed on the display, the present invention is applicable to any number of simultaneously displayed ultrasonograms.

It is also to be understood that, although Embodiment 1 has been described for the case of comparing a real-time image with a recorded image, only recorded images may be simultaneously displayed on the display.

### INDUSTRIAL APPLICABILITY

The ultrasonic diagnostic apparatus of the present invention as configured above enables setting an appropriate screen layout. As a result, the ultrasonic diagnostic apparatus can be provided that displays ultrasonograms with uncompromised visibility while requiring no effort of an operator.

### REFERENCE SIGNS LIST

1 controller
2 transmission section
3 reception section
4 image processing section
5 recording section
6 display processing section
7 control section
61 ultrasonogram output section
62 display range selection section
63 screen layout setting section
64 image combining and outputting section
100 ultrasonic diagnostic apparatus
101 probe
102 display
103 input unit
201 real-time image
202 recorded image
203 first display range
204 second display range
205 ultrasonogram display area
206 first display area
207 second display area
208 ROI

1. An ultrasonic diagnostic apparatus connectable to a display and allowing a first ultrasonogram and a second ultrasonogram to be simultaneously displayed on the display, comprising:
a display range selection section that sets a first display range on the first ultrasonogram and sets a second display range on the second ultrasonogram on the basis of the first display range set on the first ultrasonogram;
a screen layout setting section that sets a screen layout such that an ultrasonogram display area on a display screen is vertically or horizontally divided into a first display area and a second display area; and
an image combining and outputting section that generates a display screen on which the first ultrasonogram in the first display range is assigned to the first display area and the second ultrasonogram in the second display range is assigned to the second display area, and that outputs the generated display screen to the display, wherein
the screen layout setting section sets a screen layout such that the ultrasonogram display area is divided in a dividing direction that allows the first ultrasonogram in the first display range to be displayed larger in the first display area.

2. The ultrasonic diagnostic apparatus according to claim 1, wherein the screen layout setting section selects the dividing direction as a direction that allows the first ultrasonogram in the first display range to be larger when enlarged to the maximum within the first display area, and sets a screen layout such that the ultrasonogram display area is divided in the selected dividing direction.

3. The ultrasonic diagnostic apparatus according to claim 1, wherein
the second display range represents the same image region as the first display range, and
the display range selection section sets the second display range on the second ultrasonogram at a position corresponding to the position of the first display range set on the first ultrasonogram.

4. The ultrasonic diagnostic apparatus according to claim 1, wherein the screen layout setting section sets a screen layout equally divided into the first display area and the second display area.

5. The ultrasonic diagnostic apparatus according to claim 1, wherein the first display range partially or entirely covers the first ultrasonogram.

6. The ultrasonic diagnostic apparatus according to claim 1, wherein
the first ultrasonogram is a real-time image, and
the second ultrasonogram is a recorded image.

7. The ultrasonic diagnostic apparatus according to claim 1, wherein the image combining and outputting section adjusts the sizes of the first ultrasonogram in the first display range and the second ultrasonogram in the second display range to match each other.

8. The ultrasonic diagnostic apparatus according to claim 7, wherein the image combining and outputting section enlarges the first ultrasonogram in the first display range and the second ultrasonogram in the second display range within the first display area and the second display area, respectively.
